(19) United States
(12) Patent Application Publication
(10) Pub. No.: US 2026/0243103 A1
(43) Pub. Date: Aug. 20, 2026

(54) APPARATUS FOR COMMUNICATING ULTRAVIOLET C SELF-STERILIZING GERMICIDAL LIGHT TO DOOR ACCESS DEVICES AND OTHER HAND-TOUCHED DEVICES FOR HAND SANITATION

(71) Applicant: Tom Chi, Wichita, KS (US)

(72) Inventor: Tom Chi, Wichita, KS (US)

(21) Appl. No.: 18/995,128

(22) PCT Filed: Jul. 22, 2024
(86) PCT No.: PCT/US2024/038999

§ 371 Date: Jan. 15, 2025

Related U.S. Application Data
(60) Provisional application No. 63/527,845, filed on Jul. 20, 2023.

(51) Int. Cl. E05B 1/00; A61L 2/104; A61L 2/26; A61L 103/80

(52) U.S. Cl. CPC E05B 1/0069; A61L 2/104; A61L 2/26; A61L 2103/80; A61L 2202/11

(57) ABSTRACT

An apparatus for providing ultraviolet C self-sterilizing germicidal light to hand-touched devices for hand sanitation, includes a hand-touchable surface, a UVC-LED emitter for emitting ultraviolet C sterilizing germicidal light to the hand-touchable surface, and an RF4 flex board assembly having a RF4 flex board, a main printed circuit board and a flexible extension connected to the UVC-LED emitter. The main circuit board with the UVC-LED emitter being connected with the flexible extension and extending to the hand-touchable surface for initiating the UVC-LED emitter for providing germicidal light to the hand-touchable surface for sterilizing the hand-touchable surface when the hand-touchable surface is touched by the hand of a person.

### CROSS-REFERENCE TO RELATED APPLICATION

The inventor claims priority on the basis of U.S. Provisional Patent Application No. 63/527,845, filed Jul. 20, 2023, the entire disclosure of which shall be deemed to be incorporated by reference herein.

### BACKGROUND OF THE INVENTION

### Technical Field of the Invention

The present invention relates generally to disinfecting and sterilizing door access devices, such as door handles, switch plates, push-pull surfaces and alike that are touched by a user's hand by incorporating into the devices a source of ultraviolet C self-sterilizing germicidal light.

It is well established that germs from hands are easily communicated to a person's face especially when the hands touch the face several times a minute, as for example the eyes are rubbed, the nose is touched and other times when the hands consciously and unconsciously touch the face.

Since the exposed face is more than any other part of the human body highly susceptible to transmitting germs to the body's systems it imperative to maintain as best as possible a germ-free face and hands that are free of pathogens which can enter the eyes, ears, mouth, nose etc.

### Description of the Prior Art

The prior art known to the inventor pertaining to UV sanitizing apparatuses and methods includes Visco et al., U.S. Pat. No. 4,856,140, issued Aug. 15, 1989; Manson et al., U.S. Pat. No. 6,289,557, issued Sep. 18, 2001; Jones, U.S. Pat. No. 7,175,807, issued Feb. 13, 2007; Sassoon, U.S. Pat. No. 7,360,674, issued Apr. 22, 2008; Stropkay et al., U.S. Pat. No. 7,458,742, issued Dec. 2, 2008; Jones, U.S. Pat. No. 7,598,501, issued Oct. 6, 2009; Balzano, U.S. Pat. No. 8,353,085, issued Jan. 15, 2013; Caron, U.S. Pat. No. 8,375,521, issued Feb. 19, 2013; Bracey, U.S. Pat. No. 9,051,757, issued Jun. 9, 2015; Brennan, U.S. Pat. No. 9,200,771, issued Dec. 1, 2015; Roberts et al., U.S. Pat. No. 9,376,833, issued Jun. 28, 2016; and Strombeck et al., U.S. Pat. No. 10,422,158, issued Sep. 24, 2019.

The foregoing prior art patents and published patent applications generally disclose antimicrobial apparatuses and methods for sanitizing a surface, such as door handles, but unlike the present invention, do not adequately address the protection of a person's face from infection after accessing a surface, such as a door handle, which implicates different considerations.

It is thus desirable to provide a system to prevent contamination of the face and the implications of transmitting germs to the face.

### SUMMARY OF THE INVENTION

It is therefore an object of the present invention is to provide an apparatus for sanitizing door access devices such as door handles, switch plates, push-pull surfaces to prevent transmission of pathogens from door handles to a person's hand and subsequently to a person's face.

A further object of the present invention is to provide means to disinfect door handles, common switch plates, push-pull surfaces for the prevention of pathogens to contact a person and spread disease and other unsanitary conditions.

An additional object of the present invention is to utilize LED as a source of ultraviolet C self-sterilizing germicidal light for door lever and other surface sanitization.

As used throughout the within specification UVC represents (ultraviolet C light) and LED represents (light emitting diode.)

The foregoing and related objects are achieved by the present invention, which provides an apparatus for communicating ultraviolet C self-sterilizing germicidal light to door access devices and other hand-touched devices and surfaces for hand sanitation, and which includes one or more UVC-LEDs emitting sources pointed toward a door access device, or other surfaces, that are touched by a person's hands; the present invention is not limited to use with solely door handles, but may include other surfaces, such as, for example, switch plates and/or push-pull door access devices.

The present invention further includes a printed circuit board (“PCB”) and an FR4 flex board assembly located inside the door handle (or other surface) as part of door handle, such as, e.g., under door lever cover, below a door lever, inside the door lever itself, inside an end cap, etc., and may be located on one or more ends of the door handle or other proximate surface.

A power source, such as one or more batteries, may be located inside of the door handle or may be provided beneath the door lever or other another appropriate location proximate the door handle, as would be feasible. The power source, for example, batteries, may be an add-on module that snaps below the unit on the door as part of the unit. Another power source may be brought across the door jamb and supplied to the door lever to allow for continuous/intermittent operation and also enabling addition devices such as an air sanitizer to be embedded into door device body/housing.

An important feature of the apparatus of the present invention is to completely distribute LED light to each touchable surface of door lever as to ensure a complete eradication of malignant pathogens. This irradiance pattern is lacking in many devices which attempt to sanitize door levers.

An additional feature of significance of the present invention is the use of FR4 flex board to enable positioning of individual LEDs from the circuit board through to the surface of the door handle or other device, so that the UVC can irradiate the surface of the door handle (or other surface) after each touch. “FR4″ stands for ”Flame Retardant 4″ and is a material widely used in the production of rigid PCBs. More particularly, FR4 flex board is an epoxy resin laminate reinforced with fiberglass cloth to provide mechanical strength to a circuit board. The resulting combination is a robust, durable and affordable PCB suitable for a wide range of applications.

The FR4 flex board reaches from the printed circuit board to the areas where FR4 flexible extensions permit it for connecting to the surface UVC-LEDs, while the FR4 flex board remains inside the door lever just as the printed circuit board inside the lever or the lever end cap or the lever main body, etc. As used throughout the within specification “UVC” represents “ultraviolet C light” and “LED” represents “light emitting diode.”

The novel use of FR4 flex board also allows individual LEDs to penetrate the surface of the handle of the door knob, door latch or other device, while connected to the PCB located within the handle and/or handle body. This reduces the overall area of LED enclosure to each individual LEDs' space rather than a continuous and spatially cumbersome “ring” or other continuous enclosure.

Other objects and features of the present invention will become apparent when considered in combination with the accompanying drawing figures, which illustrate certain preferred embodiment of the present invention. It should, however, be noted that the accompanying drawing figures are intended to illustrate only select preferred embodiments of—5—the claimed invention and are not intended as a means for defining the limits and scope of the invention.

### BRIEF DESCRIPTION OF THE DRAWING FIGURES

In the drawing figures, wherein similar features are denoted with similar reference numerals throughout the several views:

FIG. 1 is a perspective view of the ultraviolet C self-sterilizing germicidal light emitting door access device for hand sanitation as installed on both sides of a handle with the door omitted;

FIG. 2 is a front elevational view of a common switch plate illustrating the irradiation field of the UVD-LEDs;

FIG. 2A is an additional front elevational view of a common switch plate illustrating the UVD-LEDs;

FIG. 3 is a perspective view of the FR4 flex board assembly comprising a main printed circuit board and UVC-LED emitters connected with flexible extensions to the main printed board;

FIG. 4 is a partial perspective view of a handle with LEDs on the surface thereof without a continuous surface printed circuit board connecting the LEDs;

FIG. 5 is a perspective view of a door handle with spacing in between LEDs as there is no printed circuit board connecting the LEDs on the surface of the door handle due to the FR4 flex board being located inside the door handle;

FIG. 6 is a proportional elevational view of a portion of a door handle showing how little space is required on the surface of the door handle, thereby freeing up room for a person's hand rendering cleaning much easier because only the LED and FR4 flex board penetrate the surface of the door handle;

FIG. 7 is a partial exploded perspective view of a door handle showing how the FR4 flex board is fabricated into a door handle, as well as showing the incorporation backer board and heat buffer boards;

FIG. 8 is a perspective view of a door handle with LEDs on the surface thereof, the printed circuit board being located inside of the handle; and,

FIG. 9 is a further perspective of a door handle showing the placement of LEDs on the surface thereof with the printed circuit board located inside of the handle.

### DETAILED DESCRIPTION OF THE DRAWING FIGURES AND PREFERRED EMBODIMENTS

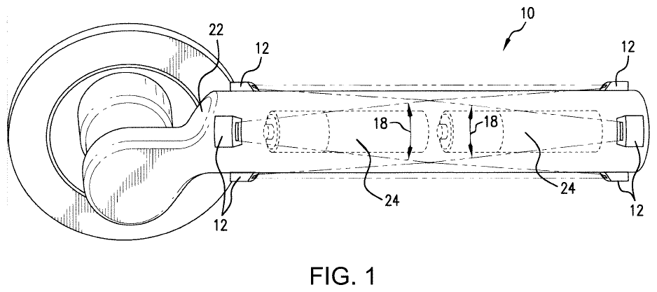

FIG. 1 illustrates invention 10 of the ultraviolet C self-sterilizing germicidal light emitting door access device for hand sanitation as installed on a typical door (not shown.) Handle 22 contains UVC-LED emitters 12, which may be positioned at either one or both ends of door handle 22. UVC-LED emitters 12, two of which are shown in FIG. 1 project two irradiation fields 18 at opposing ends of door handle 22, which together act to disinfect the surface of the door handle. FIG. 1 further shows two electric power sources 24, which may each be a battery, for powering the UVC-LED emitters 12.

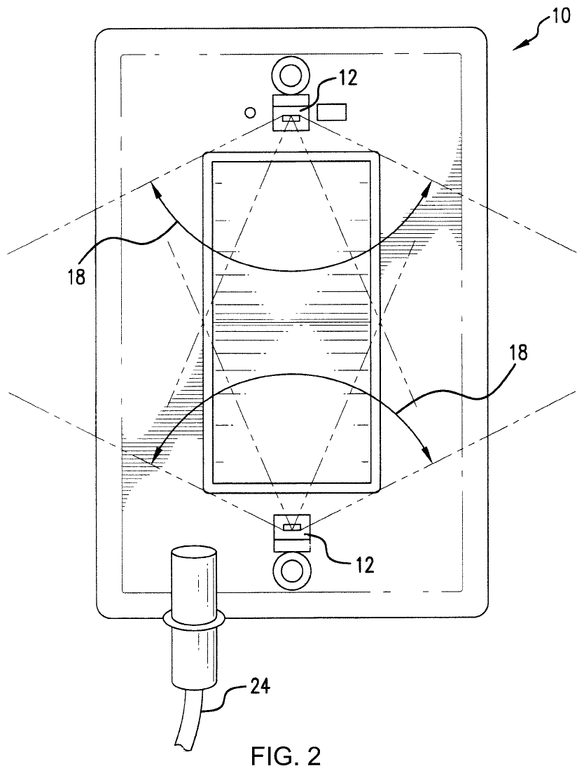

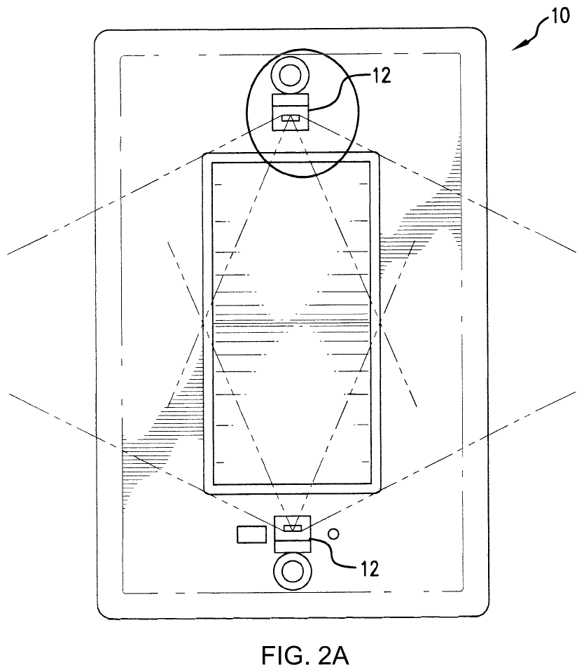

FIGS. 2 and 2A further show the present invention 10 for an alternative surface, specifically, a common switch plate for a wall switch, as having two UVC-LED emitters 12 and thereby projecting two irradiation fields 18, along with two electric power sources 24, which need not be batteries, but which may be associated with the electrical switch for the switch plate and use the location of the switch plate for providing a power source.

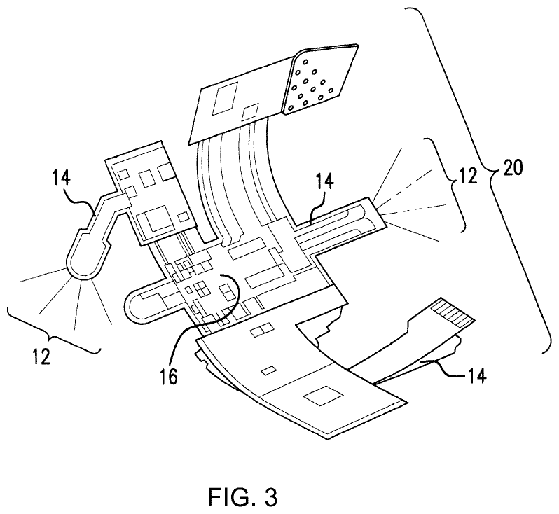

FIG. 3 is a perspective view of an FR4 flex board assembly 20 for a door handle, which includes a main printed circuit board 16 with UVC-LED emitters 12 connected with flexible extensions 14 to the main printed board 16. FR4 flex board 20 reaches from the printed circuit board 16 to the areas where FR4 flexible extensions permits it for connecting to the surface UVC-LEDs 12, while the FR4 flex board remains inside the door lever just as the printed circuit board inside the door lever or the lever end cap or the door lever main body, etc.

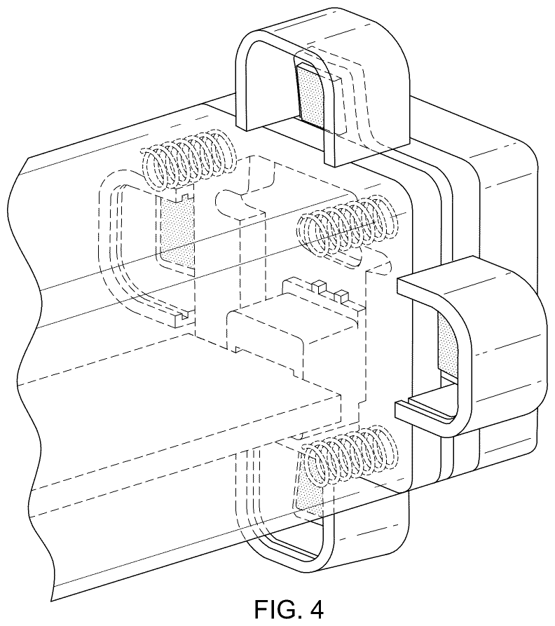

FIG. 4 is a partial perspective view of a handle with LEDs on the surface thereof without a continuous surface printed circuit board connecting the LEDs. This arrangement allows for surface positionable LEDs by passing a flex cable through to the surface from a printed circuit board located within the door handle, door lever or other device.

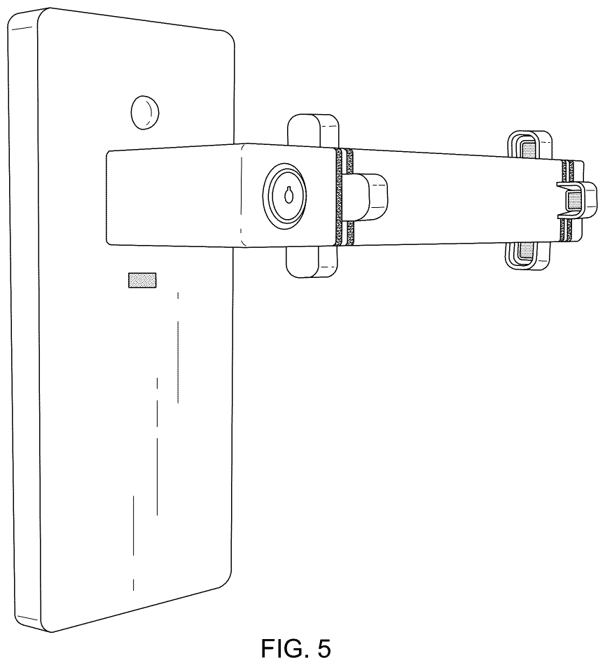

FIG. 5 is a perspective view of a door handle with spacing in between LEDs as there is no printed circuit board connecting the LEDs on the surface of the door handle due to the FR4 flex board being located inside the door handle or other device.

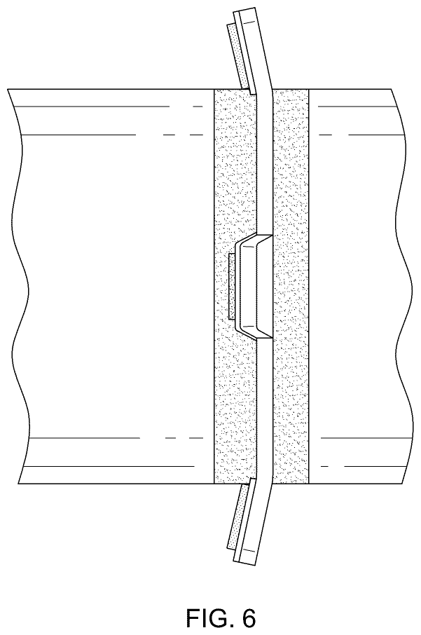

FIG. 6 is a proportional elevational view of a portion of a door handle showing how little space is required on the surface of the door handle, thereby freeing up room for a person's hand rendering cleaning much easier because only the LED and FR4 flex board penetrate the surface of the door handle. As shown in FIG. 6, only the LED (or a plurality of LEDs; not shown) and the FR4 flex board penetrate the surface of the door handle, door lever or similar device, while the printed circuit board is within a hollow portion door handle or other device.

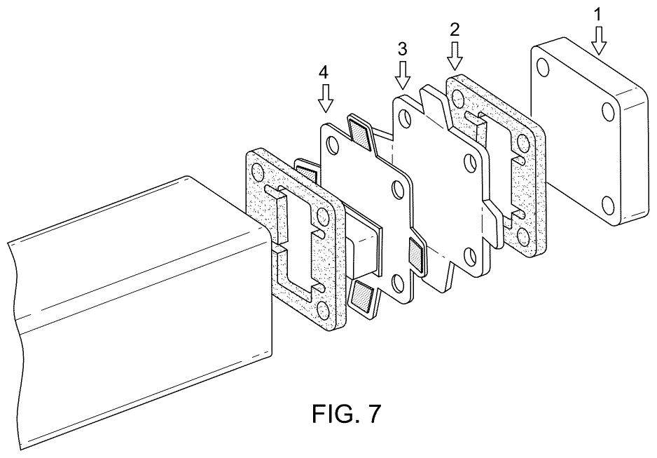

FIG. 7 is a partial exploded perspective view of a door handle showing how the FR4 flex board is fabricated into a door handle, as well as showing the incorporation backer board and heat buffer boards. In FIG. 7, the FR4 flex board is designated by reference number “1.” A flex-backer/metallic heat dispersal element is designated by reference numeral “2” so that due to a certain degree of flex with the PCB inside the door handle, it is also within the scope of the present invention to use an internal fan to aid in the dispensation of heat. Finally, reference numerals 3 and 4 in FIG. 7 denote a buffer element, such as rubber and/or a plastic for limiting heat transmission to the hand-touchable surface of the door handle, door lever or other article.

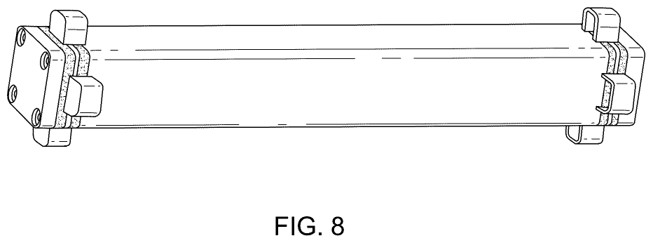

FIG. 8 is a perspective view of a door handle with LEDs on the surface thereof, the printed circuit board is located inside of the door handle or door lever.

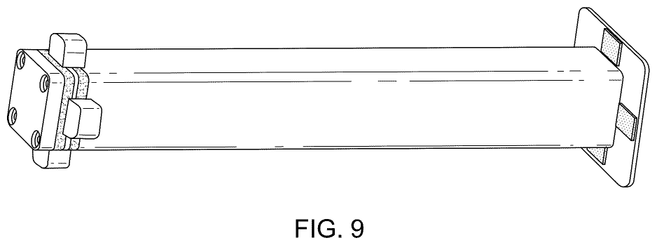

FIG. 9 is a further perspective of a door handle showing the placement of LEDs on the surface thereof with the printed circuit board located inside of the handle. FIG. 9 shows a plurality of LEDs on the surface of a door handle or door lever with the printed circuit board inside the door handle or door lever, which is possible due to the FR4 flex board.

Alternatively, the printed circuit board could be on the surface of the door handle, door lever or similar article, which is also well within the scope of the present invention.

It should be recognized that the use of FR4 flex board allows the printed circuit board to be inside or distal to the LED for allowing the LED to remain connected to the printed circuit board, even when it is pushed through an opening to the surface of the door lever or other touch device. The use of FR4 flex board allows for customizing the placement of one or more LEDs in a manner that is not possible with conventional printed circuit boards.

The use of FR4 flex board permits limiting the “footprint” of the LED mounting and protective parts. The result is that the “footprint” for the LED also become flexible as alternative sizes and/or types of compatible LEDs may be added or exchanged to suit any desired arrangement, whereas the use of simply conventional printed circuit boards limits the type and, particularly, the size and footprint of the LED, whereas with the FR4 flex board, such limitations are greatly reduced.

While only several embodiments of the present invention have been shown and described, it will be obvious to those skilled in the art that many modifications may be made to the present invention without departing from the spirit and scope thereof.

### LIST OF THE REFERENCE NUMERALS IN THE DRAWING FIGURES

Turning now descriptively to the drawing figures, in which similar reference characters denote similar elements throughout the several views, the figures illustrate the use of the present invention. With regard to the reference numerals used, the following numbering is used in FIGS. 1, 2A, 2B and 3:
10 invention
12 UVC-LED emitter
14 flexible extensions
16 main printed circuit board
18 irradiation field of UVC-LED emitters
20 RF4 flex board assembly comprising:
   (a) a main printed circuit board,
   (b) UVC-LED emitters, and
   (c) flexible extensions
22 door handle
24 electric power source

With regard to the reference numerals used, the following numbering is used in FIG. 7:
1 FR4 flex board
2 flex-backer/metallic heat dispersal element
3 heat buffer transmission element
4 heat buffer transmission element

1. An apparatus for providing ultraviolet C self-sterilizing germicidal light to hand-touched devices for providing a sanitized touch surface to ensure sanitary hand contact, comprising:
a hand-touchable surface having an area adjacent to said hand-touchable surface with an opening between said hand-touchable surface and said area;
a UVC-LED emitter for emitting ultraviolet C sterilizing germicidal light to said hand-touchable surface; and,
an FR4 flex board assembly comprising FR4 flex board, a main printed circuit board and a flexible extension connected to said UVC-LED emitter located in said area of said hand-touchable surface, said main circuit board with said UVC-LED emitter being connected with said flexible extension of said FR4 flex board and extending to said hand-touchable surface via the opening in said hand-touchable surface for initiating said UVC-LED emitter for providing germicidal light to said hand-touchable surface for sterilizing said hand-touchable surface after said hand-touchable surface is touched by a hand of a person, said FR4 flex board allowing said UVC-LED emitter to remain connected to said printed circuit board as said FR4 flex board is pushed by a user through the opening of said hand-touchable surface.

2. The apparatus for providing ultraviolet C self-sterilizing germicidal light to hand-touched devices for providing a sanitized touch surface to ensure sanitary hand contact according to claim 1, wherein said hand-touchable surface is an article for opening a door.

3. The apparatus for providing ultraviolet C self-sterilizing germicidal light to hand-touched devices for providing a sanitized touch surface to ensure sanitary hand contact according to claim 2, wherein said article for opening a door has said FR4 flex board assembly within said article for opening a door and with said UVC-LED emitter able to irradiate said hand-touchable surface of said article for opening a door.

4. The apparatus for providing ultraviolet C self-sterilizing germicidal light to hand-touched devices for providing a sanitized touch surface to ensure sanitary hand contact according to claim 3, wherein said article for opening a door is a touch-operable device.

5. The apparatus for providing ultraviolet C self-sterilizing germicidal light to hand-touched devices for providing a sanitized touch surface to ensure sanitary hand contact according to claim 4, wherein said touch-operable device is a door knob.

6. The apparatus for providing ultraviolet C self-sterilizing germicidal light to hand-touched devices for providing a sanitized touch surface to ensure sanitary hand contact according to claim 4, wherein said touch-operable device is a door lever.

7. The apparatus for providing ultraviolet C self-sterilizing germicidal light to hand-touched devices for providing a sanitized touch surface to ensure sanitary hand contact according to claim 1, further comprising a plurality of said UVC-LED emitters for irradiating a plurality of regions of said hand-touchable surface.

8. The apparatus for providing ultraviolet C self-sterilizing germicidal light to hand-touched devices for providing a sanitized touch surface to ensure sanitary hand contact according to claim 1, wherein said printed circuit board is located inside of said hand-touchable surface for a reduced footprint of said hand-touchable surface.

9. The apparatus for providing ultraviolet C self-sterilizing germicidal light to hand-touched devices for providing a sanitized touch surface to ensure sanitary hand contact according to claim 8, wherein said UVC-LED emitter is a positionable LED having a flex cable passing from said printed circuit board through said hand-touchable surface.
